(19) United States
(12) Patent Application Publication
(10) Pub. No.: US 2016/0131669 A1
(43) Pub. Date: May 12, 2016

(54) VISUALIZED BIOCHIP AND METHOD FOR SIMULTANEOUSLY DETECTING A VARIETY OF ANTIBIOTICS, ILLEGAL ADDITIVES AND BIOTOXINS

(71) Applicant: NANJING XIANGZHONG BIOTECHNOLOGY CO., LTD, Nanjing, Jiangsu (CN)

(72) Inventors: Zhonghui Li, Nanjing, Jiangsu (CN); Zhoumin Li, Nanjing, Jiangsu (CN); Siyun Sun, Nanjing, Jiangsu (CN)

(21) Appl. No.: 14/893,756

(22) PCT Filed: May 30, 2014
(86) PCT No.: PCT/CN2014/079026

§ 371 Date: Nov. 24, 2015

(30) Foreign Application Priority Data
May 31, 2013 (CN) 201310213220.8

(51) Int. Cl. G01N 33/94

(52) U.S. Cl. CPC G01N 33/9446; G01N 33/94

(57) ABSTRACT

The present invention provides a biochip for simultaneously detecting a variety of antibiotics, illegal additives and biotoxins in a visualized manner, wherein the biochip comprises a chip carrier fixed with a group of detection target antigens, the detection targets are the antibiotics, the illegal additives and the biotoxins, and the biochip is prepared by the following method: enabling the bovine serum albumin and the detection target antigens to perform sample application operation on the chip carrier through a biochip preparation system by taking bovine serum albumin as blank control, and then fixing in a water bath at 20-37° C. for 0.5-4 h for preparation. The invention further provides a method for simultaneously detecting a variety of antibiotics, illegal additives and biotoxins by using the biochip. The biochip has the advantages of simple structure, simple preparation process, low cost, multiple targets, high accuracy, high sensitivity, high precision, short detection time, simpleness and easiness in operation and no need of expensive detection instruments and is applicable to on-site large-scale primary screening of samples.

### FIELD OF THE INVENTION

The present invention belongs to the technical field of immunoassay for food safety supervision or food analysis, in particular relates to a method for simultaneously detecting residues of a variety of antibiotics, illegal additives and biotoxins in a visualized manner.

### BACKGROUND OF THE INVENTION

Antibiotics are one class of secondary metabolites which are produced by microbes (including bacteria, fungi and actinomyces) or higher animals and plants in a living process and have anti-pathogen activity or other activities. They can interfere with other chemical substances with life cell development function, and the existing common antibiotics have extracts in microbe culture solutions and compounds which are synthesized or semi-synthesized by chemical methods. The antibiotics are widely used as the antibiotics and feed additives, and may cause the residues of the antibiotics in animal derived foods. For milk and other dairy products, the residues of the antibiotics can affect the quality of the dairy products, such as seriously affecting the production of the fermented dairy products, lowering the yield, destroying the special flavor of the products in a late stage and causing economic loss to producers; and in addition, the residues of the antibiotics can cause great harm to human bodies, such as causing allergic reactions of the human bodies (such as, rash, anaphylactic shock), inhibiting the growth of normal flora in intestines, realizing massive proliferation of pathogenic bacteria and candida albicans, further causing systemic or local infections, further enabling the human bodies to produce drug resistance against the antibiosis and causing incalculable harm to clinical treatment. At present, the residues of the antibiotics in the milk, honey, poultry and aquatic products have drawn great attention of consumers, so that it is particularly important to strengthen the detection of the residues of the antibiotics. Artificially synthesized or natural substances which are added into the foods to improve the quality, the color, the smell and the taste of the foods and meet the requirements of corrosion prevention, preservation and processing technologies are called as food additives, but excess intake of the food additives and the prohibited additives is very harmful to human health. For example, melamine is a triazine nitrogen-containing heterocyclic organic compound, which is an important nitrogen heterocyclic organic chemical raw material, is relatively stable under normal circumstances, but may liberate cyanides at high temperature. As the nitrogen content in the molecular formula of the melamine is about 66%, while the average nitrogen content in proteins is about 16%, it is commonly used as the feed additive by some illegal dealers to upgrade the protein content index in food detection. Thus, the melamine is commonly known as ‘protein essence’. ‘Sudan red’ is a chemical dye, a compound called as naphthalene is contained in chemical components of the sudan red, the substance has an azo structure, and the nature of the chemical structure determines that it has carcinogenicity and has obvious toxicity to liver and kidney organs of the human bodies. In 1995, the European Union (EU) and other countries banned the addition of the sudan red into the foods as a pigment, and then China also banned expressly.

Biotoxins are toxic substances produced by various organisms (animals, plants and microbes), are natural toxins and refer to organism derived toxic chemical substances incapable of performing self-reproduction, including various chemical substances which are produced by the animals, the plants and the microbes and are toxic and harmful to other biological species. More than 300 species of mycotoxins have been found so far, wherein more than 20 species have been separated and proved to be toxic. The food-related mycotoxins comprise: aflatoxin, sterigmatocystin, islanditoxin, luteoskyin, citrinin, patulin, trichothecenes, zearalenone, butenolide and the like. The mycotoxins are significantly harmful to the human bodies and can cause liver, kidney and nerve intoxication of the human bodies and initiate severe dermatitis. For example, the aflatoxin is designated as class 1 carcinogen by the World Health Organization, the toxicity is far higher than that in the cyanides, arsenides and organic farm chemicals, wherein B1 has highest toxicity which is 68 times higher than that in white arsenic, is second only to botulinum toxin and has the strongest toxicity in the existing known molds. B1 is the most dangerous carcinogen, and can be often detected in corns, peanuts, cotton seeds and some dried fruits, wherein the pollution of the peanuts and the corn is most serious. M1 is a metabolite of B1 and mainly exists in the milk.

At present, the common detection methods for the residues of the antibiotics, illegal additives and biotoxins at home and abroad are mainly divided into the following two categories: physical and chemical detection methods and immunoassay methods. The physical and chemical detection methods are the methods for determining the content by utilizing special reactions or natures of groups in molecules of the antibiotics, such as high performance liquid chromatography, gas chromatography, colorimetry and fluorescence spectrophotometry, wherein the high performance liquid chromatography (HPLC) and chromatography-mass spectrometry (TLC-MS, GC-MS, LC-MS and the like) are the most commonly used methods. The physical and chemical detection methods have the shortcomings of high equipment requirements, high technical difficulty, high requirements on technical staff, minute and complicated operation and need of performing a lot of complex sample pretreatment operations, and thus is not suitable for on-site monitoring and screening of a large number of samples. The immunoassay methods are analysis methods for determining the content of the antibiotics based on antigen and antibody identification or receptor and ligand identification, such as enzyme-linked immunosorbent assay (ELISA) and receptor adsorption assay. These methods have the advantages of high sensitivity, strong specificity and simple operation. However, the existing immunoassay methods, such as ELISA, cannot simultaneously determine multiple targets. For example, if a variety of antibiotics in a sample need to be determined, multiple tests need to be performed, so that more time and materials need to be consumed. In addition, for the detection against the antibiotics, microbe methods are also used frequently. The microbiology detection methods are the methods for qualitatively or quantitatively determining the residues of the antibiotics in the sample by utilizing the inhibition effects of the antibiotics against physiological functions and metabolism of microbes, such as a microbial growth inhibition method, a microbial receptor method and an enzyme colorimetry method. These methods require heating equipment to culture the microbes, and the microbe methods consume very long time and can not perform fast detection. Thus, it is urgent to develop a sensitive, accurate and fast detection method which can simultaneously detect a variety of antibiotics, illegal additives and biotoxins.

### SUMMARY OF THE INVENTION

### Objects of the Invention

The first object of the invention is to provide a biochip for simultaneously detecting a variety of antibiotics, illegal additives and biotoxins in a visualized manner, and the second object of the invention is to provide a method for simultaneously detecting residues of a variety of antibiotics, illegal additives and biotoxins in a visualized manner by utilizing the biochip, which can make up for the blank of a technology for simultaneously and quantitatively detecting the antibiotics, the illegal additives and the biotoxins in the prior art, meet the requirements of simultaneously performing fast detection on the variety of the antibiotics, the illegal additives and the biotoxins in animal derived foods, and have the characteristics of multiple targets, low cost, high sensitivity, short detection time, simpleness and easiness in operation and the like.

### Technical Solutions

A biochip for simultaneously detecting a variety of antibiotics, illegal additives and biotoxins in a visualized manner comprises a chip carrier fixed with a group of detection target antigens, the detection targets comprise the antibiotics, the illegal additives and the biotoxins, and the biochip is prepared by the following method: enabling the bovine serum albumin and the detection target antigens to perform sample application operation on the chip carrier through a biochip preparation system by taking bovine serum albumin as blank control, and then fixing in a water bath at 20-37° C. for 0.5-4 h for preparation.

Wherein, the biochip is constituted by a group of matrices constituted by the detection target antigens, and the number of columns of each matrix is set to be a multiple of 3; and the number of the columns of each matrix is set to be the multiple of 3, so that each detection target antigen can point three repetition points to enable the determination result to be more intuitive and more accurate.

Wherein, the chip carrier is a 96-well plate, a 384-well plate, a transparent high polymer chip base, a film or a glass sheet.

Wherein, the β-lactam antibiotics include penicillin antibiotics and cephalosporin antibiotics; the aminoglycoside antibiotics include gentamicin, kanamycin, streptomycin and neomycin; the tetracycline antibiotics include tetracycline, terramycin, aureomycin and doxycycline; the macrolide antibiotics include erythromycin, clarithromycin and roxithromycin; the sulfonamide antibiotics include sulfadiazine, sulfamethazine, sulfadimethoxine, sulfamethoxazole and sulfadoxine; the quinolone antibiotics include norfloxacin, ciprofloxacin, ofloxacin, gatifloxacin, sparfloxacin and moxifloxacin; the nitrofuran antibiotics include furazolidone, furaltadone, nitrofurantoin and nitrofurazone; and other antibiotics include chloramphenicol, thiamphenicol, clindamycin and lincomycin. The antibiotics described in the invention are not limited to the specific antibiotics listed above, and the existing antibiotics in the prior art can adopt the method of the invention to detect the content.

Wherein, the illegal additives are one or more of melamine, malachite green and sudan red.

Wherein, the biotoxins are one or more of aflatoxin, T-2 toxin, vomitoxin, zearalenone, fumonisin, ochrotoxin A, sterigmatocystin, butenolide, citrinin, patulin, trichothecenes and botulinum toxin; and specifically, the aflatoxin is aflatoxin M1, M2, B1, B2, G1 or G2. The biotoxins described in the invention are not limited to the specific biotoxins listed above, and the existing biotoxins in the prior art can adopt the method of the invention to detect the content.

The present invention further provides a method for simultaneously detecting residues of a variety of antibiotics, illegal additives and biotoxins in a visualized manner, comprising the following steps:

(1) respectively adding mixed standard solutions containing different concentration gradients of detection targets into part of reaction wells of a biochip, respectively adding a solution of a sample to be detected into the remaining reaction wells, further sequentially adding monoclonal antibodies and labeled goat anti-mouse IgG corresponding to the detection targets into all the reaction wells, reacting in a water bath at 20-37° C. for 10-20 min and washing with washing liquid;

(2) further adding a color developing agent into all the reaction wells, performing color development reaction for 2-5 min, and then terminating the reaction; and

(3) performing image acquisition by using CCD, making a standard curve according to gray scale values of antigens coated by the detection targets and concentration logarithms of the standard solutions, and respectively calculating the contents of the detection targets in the sample solution.

Wherein, the sample to be detected is an animal derived food, including milk, milk powder, cheese, feed, urine, animal tissues (such as pork, beef, chicken, pork liver or chicken liver or the like), serum, honey, royal jelly, eggs and aquatic products (such as fish or shrimp or the like).

Wherein, the preparation method of the solution of the sample to be detected is as follows: when the sample to be detected is liquid, taking 50 μL-1 mL and putting into a 10 mL EP pipe, and diluting with a buffer solution for 10-100 times to obtain the solution of the sample to be detected; and when the sample to be detected is solid, taking 5 g of the homogeneous sample, adding 8 mL of the buffer solution, mixing for 2-5 min, putting into a water bath at 50° C. for 10-30 min, centrifuging for 5-10 min, taking 50 μL of supernatant liquid, adding 450 μL of the buffer solution, uniformly mixing, and taking 50 μL-500 μL to obtain the solution of the sample to be detected. Wherein, in step (1), the adding volume of the mixed standard solution of each detection target is 10-200 μL; the adding volume of the sample to be detected is the same as that of a standard solution of free antibiotics; the amount of the monoclonal antibody added in each reaction well is 10-200 μL; the amount of the labeled goat anti-mouse IgG added in each reaction well is 10-200 μL; and the amounts of the monoclonal antibodies added in the different reaction wells are the same, and the amounts of the labeled goat anti-mouse IgG added in different reaction wells are the same.

Wherein, the labeled goat anti-mouse IgG is a nano-material or a biological enzyme-labeled goat anti-mouse IgG, such as gold nanoparticle (5-25 nm)-labeled goat anti-mouse IgG, silver nanoparticle (5-25 nm)-labeled goat anti-mouse IgG, horseradish peroxidase-labeled goat anti-mouse IgG or alkaline phosphatase-labeled goat anti-mouse IgG.

Wherein, the washing liquid is a buffer solution, and specifically comprises a phosphate buffer solution and a Tris buffer solution.

Wherein, in step (2), the color developing agent comprises a biological catalyst and a substrate thereof or a chemical catalyst and the substrate thereof, and the adding volume of the color developing agent is 10-100 μL. The color developing agent can be reasonably selected according to the public knowledge in this field, the biological catalysts and the substrates thereof include: the horseradish peroxidase and the substrate thereof, namely, an o-phenylenediamine/hydrogen peroxide system or a tetramethylbenzidine/hydrogen peroxide system, and the alkaline phosphatase and the substrate thereof, namely, a BCIP/NBT system; and the chemical catalysts and the substrates thereof include: nanogold or nanosilver and the substrate thereof, namely, a chloroauric acid/tannic acid system or a chloroauric acid/hydrochinone system. The biological catalysts have relatively high requirements for reaction conditions, for example, the catalysis efficiency of part of the catalysts is the highest at 37° C.; while the chemical catalysts have the advantages of high stability and a wide range of applications.

### Beneficial Effects

The biochip for simultaneously detecting the variety of the antibiotics, the illegal additives and the biotoxins in the visualized manner provided by the invention has the advantages of simple structure, simple preparation process, low cost, multiple targets, high accuracy, high sensitivity, high precision, short detection time, simpleness and easiness in operation and no need of expensive detection instruments and is applicable to on-site large-scale primary screening of samples.

The biochip fixes the detection target antigens on the chip carrier, and when in use, the solution of the sample to be detected and the monoclonal antibodies and labeled secondary antibodies corresponding to the detection targets are added into the reaction wells, so that the free antibiotics, the illegal additives and the biotoxins in the solution of the sample to be detected perform competitive reaction with the coating antigens fixed on the biochip. The more the free detection targets in the solution of the sample to be detected are, the less the reaction of the coating antigens fixed on the biochip is. After color development, spots with different shades, which can be visually examined, are formed in different positions of an array of the antigens fixed on the biochip, image acquisition is performed through a CCD, and then the simultaneous quantitative detection of the variety of the antibiotics, the illegal additives and the biotoxins can be finally realized.

Specifically, compared with the prior art, the invention has the following outstanding features:

(1) High accuracy: by using the chip provided by the invention to detect the variety of the antibiotics, the illegal additives and the biotoxins, the recovery rate against the sample added in the milk is 80%-120%, the accuracy is high, and the chip is suitable for detection of the actual samples.

(2) High sensitivity: by using the chip provided by the invention to detect the variety of the antibiotics, the illegal additives and the biotoxins, the lowest detection limit against the chloramphenicol and other antibiotic substances in the milk achieves 0.05 ng/ml, the lowest detection limit against the melamine and other illegal additives in the milk achieves 5 ng/ml, the lowest detection limit against the aflatoxin M1 and other biotoxins in the milk achieves 0.2 ng/ml, and the sensitivity is high.

(3) High precision: by using the chip provided by the invention to detect the variety of the antibiotics, the illegal additives and the biotoxins, the specificity against the antibodies of the antibiotics, the illegal additives and the biotoxins is better, the CV value between the wells is lower than 12%, the CV value in the wells is lower than 8%, and the precision is high.

(4) High efficiency: by using the chip provided by the invention to detect the variety of the antibiotics, the illegal additives and the biotoxins, the concentrations of the variety of the antibiotics, the illegal additives and the biotoxins can be quantitatively calculated according to an external standard curve. By using the biochip, the contents of the variety of harmful substances can be simultaneously measured in one test, so that the operation steps are greatly reduced, the using amount of a reagent is reduced, the cost is reduced, special expensive experimental instruments are not required, and a thought is simultaneously provided for on-site fast detection; and compared with the existing ELISA method, the invention performs quantitative detection according to the shade of the color of the spots of the coating antigens of the biochip, thereby overcoming the defect that the ELISA method can only determine the content of the single harmful substance by performing color development once in the solution. The method of the invention can detect the variety of the antibiotics, the illegal additives and the biotoxins simultaneously, quickly and quantitatively, while the existing methods can only detect one antibiotic. The reason is that internal standard substances are usually added in the existing methods, then the detection of multiple substances is interfered, and the accuracy of detection of other substances to be detected is further affected.

### BRIEF DESCRIPTION OF THE DRAWINGS

FIG. 1a-FIG. 1o are standard curves made by the method of invention.

1a is the standard curve of kanamycin; the curve of the kanamycin has good linearity in the range of 1 ng/mL-20 ng/mL, R²>0.99, the CV value between wells is lower than 12%, the CV value in the wells is lower than 8%, and the method has high precision. Furthermore, the sample recovery rate of milk is 90%-120%, and the method has high accuracy.

1b is the standard curve of cefalexin; the curve of the cefalexin has good linearity in the range of 2 ng/mL-50 ng/mL, R²>0.99, the CV value between the wells is lower than 12%, the CV value in the wells is lower than 8%, and the method has high precision. Furthermore, the sample recovery rate of the milk is 85%-105%, and the method has high accuracy.

1c is the standard curve of gentamicin; the curve of the gentamicin has good linearity in the range of 1 ng/mL-20 ng/mL, R²>0.99, the CV value between wells is lower than 12%, the CV value in the wells is lower than 8%, and the method has high precision. Furthermore, the sample recovery rate of the milk is 87%-112%, and the method has high accuracy.

1d is the standard curve of sulfamethazine; the curve of the sulfamethazine has good linearity in the range of 0.8 ng/ml/mL-20 ng/mL, R²>0.99, the CV value between wells is lower than 12%, the CV value in the wells is lower than 8%, and the method has high precision. Furthermore, the sample recovery rate of the milk is 92%-109%, and the method has high accuracy.

1e is the standard curve of tetracycline; the curve of the tetracycline has good linearity in the range of 0.05 ng/mL-1.5 ng/mL, R²>0.99, the CV value between wells is lower than 12%, the CV value in the wells is lower than 8%, and the method has high precision. Furthermore, the sample recovery rate of the milk is 85%-110%, and the method has high accuracy.

1f is the standard curve of erythromycin; the curve of the erythromycin has good linearity in the range of 5 ng/mL-150 ng/mL, R²>0.99, the CV value between wells is lower than 12%, the CV value in the wells is lower than 8%, and the method has high precision. Furthermore, the sample recovery rate of the milk is 90%-110%, and the method has high accuracy.

1g is the standard curve of ciprofloxacin; the curve of the ciprofloxacin has good linearity in the range of 0.2 ng/ml/mL-1.5 ng/mL, R²>0.99, the CV value between wells is lower than 12%, the CV value in the wells is lower than 8%, and the method has high precision. Furthermore, the sample recovery rate of the milk is 85%-110%, and the method has high accuracy.

1h is the standard curve of furaltadone; the curve of the furaltadone has good linearity in the range of 0.1 ng/mL-8 ng/mL, R²>0.99, the CV value between wells is lower than 12%, the CV value in the wells is lower than 8%, and the method has high precision. Furthermore, the sample recovery rate of milk is 80%-120%, and the method has high accuracy.

1i is the standard curve of chloramphenicol; the curve of the chloramphenicol has good linearity in the range of 0.05 ng/mL-4 ng/mL, R²>0.99, the CV value between wells is lower than 12%, the CV value in the wells is lower than 8%, and the method has high precision. Furthermore, the sample recovery rate of milk is 90%-120%, and the method has high accuracy.

1j is the standard curve of lincomycin; the curve of the lincomycin has good linearity in the range of 0.2 ng/mL-16 ng/mL, R²>0.99, the CV value between wells is lower than 12%, the CV value in the wells is lower than 8%, and the method has high precision. Furthermore, the sample recovery rate of the milk is 85%-105%, and the method has high accuracy.

1k is the standard curve of melamine; the curve of the melamine has good linearity in the range of 5 ng/mL-200 ng/mL, R²>0.99, the CV value between wells is lower than 12%, the CV value in the wells is lower than 8%, and the method has high precision. Furthermore, the sample recovery rate of the milk is 95%-105%, and the method has high accuracy.

1l is the standard curve of aflatoxin M1; the curve of the aflatoxin M1 has good linearity in the range of 0.2 ng/mL-4 ng/mL, R²>0.99, the CV value between wells is lower than 12%, the CV value in the wells is lower than 8%, and the method has high precision. Furthermore, the sample recovery rate of the milk is 90%-110%, and the method has high accuracy.

1m is the standard curve of T-2 toxin; the curve of the T-2 toxin has good linearity in the range of 20 ng/mL-250 ng/mL, R²>0.99, the CV value between wells is lower than 12%, the CV value in the wells is lower than 8%, and the method has high precision. Furthermore, the sample recovery rate of the milk is 90%-112%, and the method has high accuracy.

1n is the standard curve of zearalenone; the curve of the zearalenone has good linearity in the range of 10 ng/mL-100 ng/mL, R²>0.99, the CV value between wells is lower than 12%, the CV value in the wells is lower than 8%, and the method has high precision. Furthermore, the sample recovery rate of the milk is 85%-110%, and the method has high accuracy.

1o is the standard curve of fumonisin; the curve of the fumonisin has good linearity in the range of 2.5 ng/mL-200 ng/mL, R²>0.99, the CV value between wells is lower than 12%, the CV value in the wells is lower than 8%, and the method has high precision. Furthermore, the sample recovery rate of the milk is 90%-112%, and the method has high accuracy.

FIG. 2 is a diagram of a biochip of an embodiment of the invention; and 15 items, namely, kanamycin, cefalexin, gentamicin, sulfamethazine, tetracycline, erythromycin, ciprofloxacin, furaltadone, chloramphenicol, lincomycin, melamine, aflatoxin M1, T-2 toxin, zearalenone and fumonisin, can be simultaneously detected in each reaction well of the biochip.

FIG. 3 is a diagram of the biochip after simultaneously detecting kanamycin, cefalexin, gentamicin, sulfamethazine, tetracycline, erythromycin, ciprofloxacin, furaltadone, chloramphenicol, lincomycin, melamine, aflatoxin M1, T-2 toxin, zearalenone and fumonisin in milk by adopting the invention, wherein A-F wells are standard solution reaction wells, and 1-34 wells are actual milk sample reaction wells.

### DETAILED DESCRIPTION OF THE EMBODIMENTS

Those skilled in the art can better understand the invention according to the following embodiments. However, the contents described in the embodiments are only used for describing the invention and should not and can not limit the invention described in the claims in detail.

### Embodiment 1

The contents of kanamycin, cefalexin, gentamicin, sulfamethazine, tetracycline, erythromycin, ciprofloxacin, furaltadone, chloramphenicol, lincomycin, melamine, aflatoxin M1, T-2 toxin, zearalenone and fumonisin in milk were simultaneously and quantitatively detected.

The reagents used in the experiment were as follows:

(1) Standard solutions:

The standard solutions used in the embodiment were six in total, and the composition of each was as follows:

a. Mixed standard solution 1: wherein the concentration of kanamycin was 0 ng/mL, the concentration of cefalexin was 0 ng/mL, the concentration of gentamicin was 0 ng/mL, the concentration of sulfamethazine was 0 ng/mL, the concentration of tetracycline was 0 ng/mL, the concentration of erythromycin was 0 ng/mL, the concentration of ciprofloxacin was 0 ng/mL, the concentration of furaltadone was 0 ng/mL, the concentration of chloramphenicol was 0 ng/mL, the concentration of lincomycin was 0 ng/mL, the concentration of melamine was 0 ng/mL, the concentration of aflatoxin M1 was 0 ng/mL, the concentration of T-2 toxin was 0 ng/mL, the concentration of zearalenone was 0 ng/mL and the concentration of fumonisin was 0 ng/mL.

b. Mixed standard solution 2: wherein the concentration of kanamycin was 1 ng/mL, the concentration of cefalexin was 2 ng/mL, the concentration of gentamicin was 1 ng/mL, the concentration of sulfamethazine was 0.8 ng/mL, the concentration of tetracycline was 0.05 ng/mL, the concentration of erythromycin was 5 ng/mL, the concentration of ciprofloxacin was 0.2 ng/mL, the concentration of furaltadone was 0.1 ng/mL, the concentration of chloramphenicol was 0.05 ng/mL, the concentration of lincomycin was 0.2 ng/mL, the concentration of melamine was 5 ng/mL, the concentration of aflatoxin M1 was 0.2 ng/mL, the concentration of T-2 toxin was 20 ng/mL, the concentration of zearalenone was 10 ng/mL and the concentration of fumonisin was 2.5 ng/mL.

c. Mixed standard solution 3: wherein the concentration of kanamycin was 2.5 ng/mL, the concentration of cefalexin was 5 ng/mL, the concentration of gentamicin was 2 ng/mL, the concentration of sulfamethazine was 2 ng/mL, the concentration of tetracycline was 0.15 ng/mL, the concentration of erythromycin was 15 ng/mL, the concentration of ciprofloxacin was 0.6 ng/mL, the concentration of furaltadone was 0.3 ng/mL, the concentration of chloramphenicol was 0.15 ng/mL, the concentration of lincomycin was 0.6 ng/mL, the concentration of melamine was 20 ng/mL, the concentration of aflatoxin M1 was 0.5 ng/mL, the concentration of T-2 toxin was 35 ng/mL, the concentration of zearalenone was 20 ng/mL and the concentration of fumonisin was 7.5 ng/mL.

d. Mixed standard solution 4: wherein the concentration of kanamycin was 5 ng/mL, the concentration of cefalexin was 10 ng/mL, the concentration of gentamicin was 4 ng/mL, the concentration of sulfamethazine was 4 ng/mL, the concentration of tetracycline was 0.3 ng/mL, the concentration of erythromycin was 30 ng/mL, the concentration of ciprofloxacin was 1.8 ng/mL, the concentration of furaltadone was 0.9 ng/mL, the concentration of chloramphenicol was 0.45 ng/mL, the concentration of lincomycin was 1.8 ng/mL, the concentration of melamine was 50 ng/mL, the concentration of aflatoxin M1 was 1 ng/mL, the concentration of T-2 toxin was 75 ng/mL, the concentration of zearalenone was 40 ng/mL and the concentration of fumonisin was 20 ng/mL.

e. Mixed standard solution 5: wherein the concentration of kanamycin was 10 ng/mL, the concentration of cefalexin was 25 ng/mL, the concentration of gentamicin was 10 ng/mL, the concentration of sulfamethazine was 10 ng/mL, the concentration of tetracycline was 0.6 ng/mL, the concentration of erythromycin was 60 ng/mL, the concentration of ciprofloxacin was 5.4 ng/mL, the concentration of furaltadone was 2.7 ng/mL, the concentration of chloramphenicol was 1.35 ng/mL, the concentration of lincomycin was 5.4 ng/mL, the concentration of melamine was 100 ng/mL, the concentration of aflatoxin M1 was 2 ng/mL, the concentration of T-2 toxin was 125 ng/mL, the concentration of zearalenone was 60 ng/mL and the concentration of fumonisin was 60 ng/mL.

f. Mixed standard solution 6: wherein the concentration of kanamycin was 20 ng/mL, the concentration of cefalexin was 50 ng/mL, the concentration of gentamicin was 20 ng/mL, the concentration of sulfamethazine was 20 ng/mL, the concentration of tetracycline was 1.5 ng/mL, the concentration of erythromycin was 150 ng/mL, the concentration of ciprofloxacin was 16 ng/mL, the concentration of furaltadone was 8 ng/mL, the concentration of chloramphenicol was 4 ng/mL, the concentration of lincomycin was 16 ng/mL, the concentration of melamine was 200 ng/mL, the concentration of aflatoxin M1 was 4 ng/mL, the concentration of T-2 toxin was 250 ng/mL, the concentration of zearalenone was 100 ng/mL and the concentration of fumonisin was 200 ng/mL.

The above compound standard products were purchased from the National Institute for the Control of Pharmaceutical and Biological Products, and when in use, the compound standard products were weighed and diluted with pure water to the desired concentration.

(2) Sample solution: the sample solution was commercially available milk and was negative according to HPLC detection, and the standard solution was added into the sample solution to the desired concentration.

(3) Monoclonal antibodies:

a. The monoclonal antibody corresponding to the kanamycin was mouse anti-kanamycin monoclonal antibody, purchased from Abcam Company.

b. The monoclonal antibody corresponding to the cefalexin was mouse anti-cefalexin monoclonal antibody, purchased from Abcam Company.

c. The monoclonal antibody corresponding to the gentamicin was mouse anti-gentamicin monoclonal antibody, purchased from Pierce Company.

d. The monoclonal antibody corresponding to the sulfamethazine was mouse anti-sulfamethazine monoclonal antibody, purchased from Biodesign Company.

e. The monoclonal antibody corresponding to the tetracycline was mouse anti-tetracycline monoclonal antibody, purchased from Santa Cruz Company.

f. The monoclonal antibody corresponding to the erythromycin was mouse anti-erythromycin monoclonal antibody, purchased from Sigma-Aldrich Company.

g. The monoclonal antibody corresponding to the ciprofloxacin was mouse anti-ciprofloxacin monoclonal antibody, purchased from Abcam Company.

h. The monoclonal antibody corresponding to the furaltadone was mouse anti-furaltadone monoclonal antibody, purchased from AbMart Company.

i. The monoclonal antibody corresponding to the chloramphenicol was mouse anti-chloramphenicol monoclonal antibody, purchased from eBioscience Company.

j. The monoclonal antibody corresponding to the lincomycin was mouse anti-lincomycin monoclonal antibody, purchased from Hangzhou Biotechnology Co., Ltd.

k. The monoclonal antibody corresponding to the melamine was mouse anti-melamine monoclonal antibody, purchased from Biodesign Company.

l. The monoclonal antibody corresponding to the aflatoxin M1 was mouse anti-aflatoxin M1 monoclonal antibody, purchased from R-Biopharm Company.

m. The monoclonal antibody corresponding to the T-2 toxin was mouse anti-T-2 toxin monoclonal antibody, purchased from Biodesign Company.

n. The monoclonal antibody corresponding to the zearalenone was mouse anti-zearalenone monoclonal antibody, purchased from Sigma-Aldrich Company.

o. The monoclonal antibody corresponding to the fumonisin was mouse anti-fumonisin monoclonal antibody, purchased from Hangzhou Biotechnology Co., Ltd.

(4) Labeled goat anti-mouse IgG was colloidal gold (25 nm)-labeled goat anti-mouse IgG, could be combined with the above six antibodies and was purchased from Sigma-Aldrich Company.

(5) Color developing agent: the color developing agent used in the reaction comprised nanogold and a substrate thereof, namely, 0.4M of HAuCl₄ and 0.1M of tannic acid in the volume ratio of 1:1, wherein the HAuCl₄ and the tannic acid were purchased from Sigma-Aldrich Company.

(6) Formulas of buffer solutions were respectively as follows:

The formula of a phosphate buffer solution comprised 135 mM of NaCl, 2.7 mM of KCl, 1.5 mM of KH₂PO₄, and 8 mM of K₂HPO₄, and the pH was regulated to 7.4. The formula of a Tris buffer solution comprised 50 ml of tris(hydrosymenthyl)-aminomethande (Tris) with the concentration of 0.1 mol/L and 44.7 ml of hydrochloric acid with the concentration of 0.1 mol/L, and the Tris buffer solution was diluted with water till 100 ml.

The detection steps were as follows:

(1) preparation of a biochip: bovine serum albumin was taken as blank control, a biochip preparation instrument (US BioDot Company) was used to perform sample application on a 96-well plate to prepare a micro-array chip, bovine serum albumin, kanamycin, cefalexin, gentamicin, sulfamethazine, tetracycline, erythromycin, ciprofloxacin, furaltadone, chloramphenicol, lincomycin, melamine, aflatoxin M1, T-2 toxin, zearalenone and fumonisin antigens were subject to sample application operation on the 96-well plate, fixing was performed in a water bath at 37° C. for 0.5 h, a micro-array structure was formed on the 96-well plate, array points in different positions corresponded to different antigens; a 384-well plate, a transparent high-polymer chip base, a film or a glass sheet could also be adopted to replace the 96-well plate as a chip carrier;

(2) preparation of a solution of a sample to be detected: 100 μL of milk sample was taken and placed in a 10 mL EP pipe, and diluted with a PBS buffer solution (0.1M, with the pH of 7.4) for 10 times to obtain the solution of the sample to be detected;

(3) 50 μL of mixed standard solutions containing different concentration gradients of free kanamycin, cefalexin, gentamicin, sulfamethazine, tetracycline, erythromycin, ciprofloxacin, furaltadone, chloramphenicol, lincomycin, melamine, aflatoxin M1, T-2 toxin, zearalenone and fumonisin were respectively added into part of reaction wells of the 96-well plate, 50 μL of the solution of the sample to be detected is respectively added into each of the remaining reaction wells, 50 μL of monoclonal antibody mixed solutions corresponding to the kanamycin, cefalexin, gentamicin, sulfamethazine, tetracycline, erythromycin, ciprofloxacin, furaltadone, chloramphenicol, lincomycin, melamine, aflatoxin M1, T-2 toxin, zearalenone and fumonisin and 25 μL of nanosilver-labeled goat anti-mouse IgG were continuously added into the reaction wells added with the mixed standard solutions or the sample solution, reaction was performed in the water bath at 37° C. for 20 min and then washing was performed with PBST;

(4) 50 μL of color developing agent and a substrate thereof were continuously added into each reaction well, the use was performed immediately after preparation, the color development reaction was performed for 5 min, and then the reaction was terminated;

(5) image acquisition was performed by a CCD, and the contents of kanamycin, cefalexin, gentamicin, sulfamethazine, tetracycline, erythromycin, ciprofloxacin, furaltadone, chloramphenicol, lincomycin, melamine, aflatoxin M1, T-2 toxin, zearalenone, fumonisin and other substances in the sample were quantitatively detected by utilizing an external standard curve method according to array gray scale value signals in positions of coating antigens corresponding to the kanamycin, cefalexin, gentamicin, sulfamethazine, tetracycline, erythromycin, ciprofloxacin, furaltadone, chloramphenicol, lincomycin, melamine, aflatoxin M1, T-2 toxin, zearalenone and fumonisin.

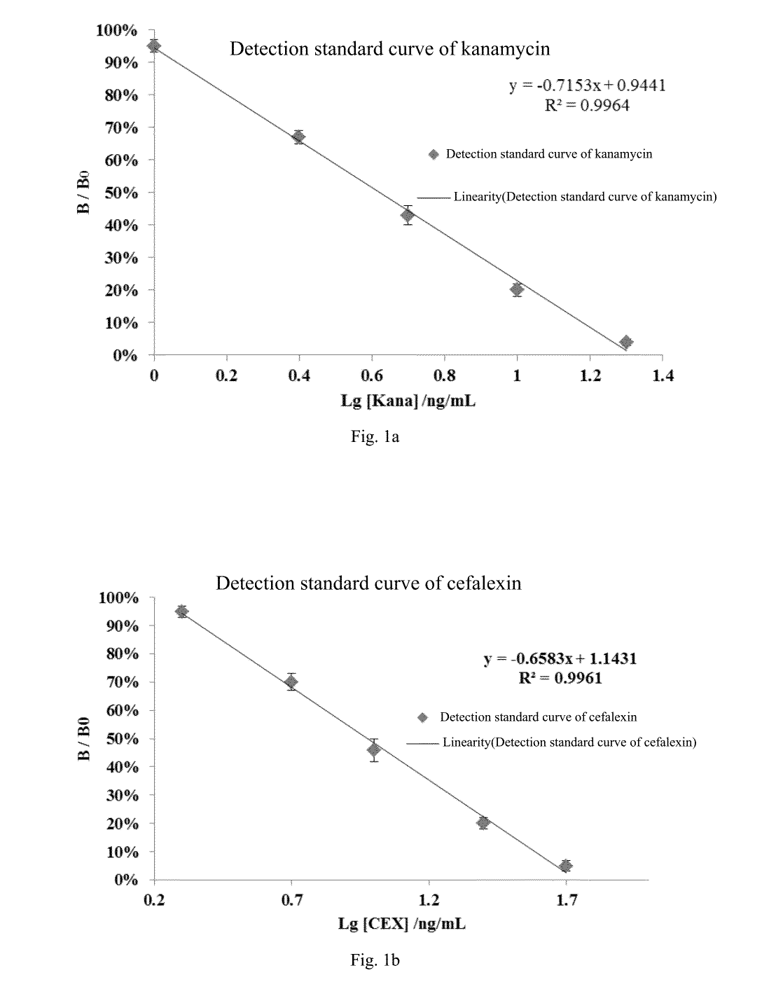

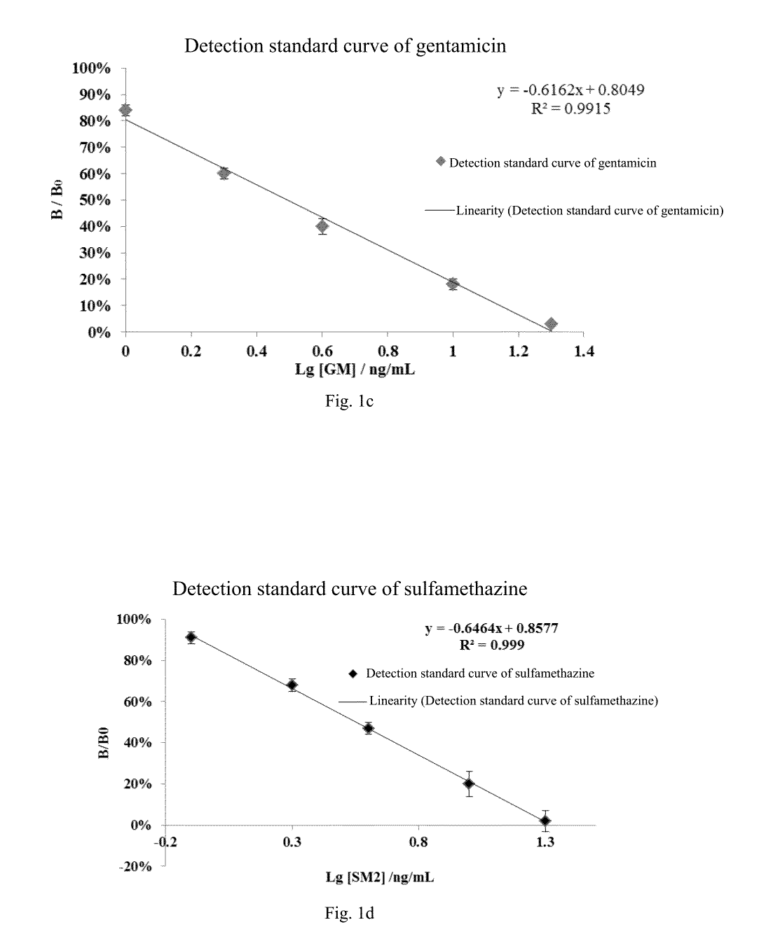

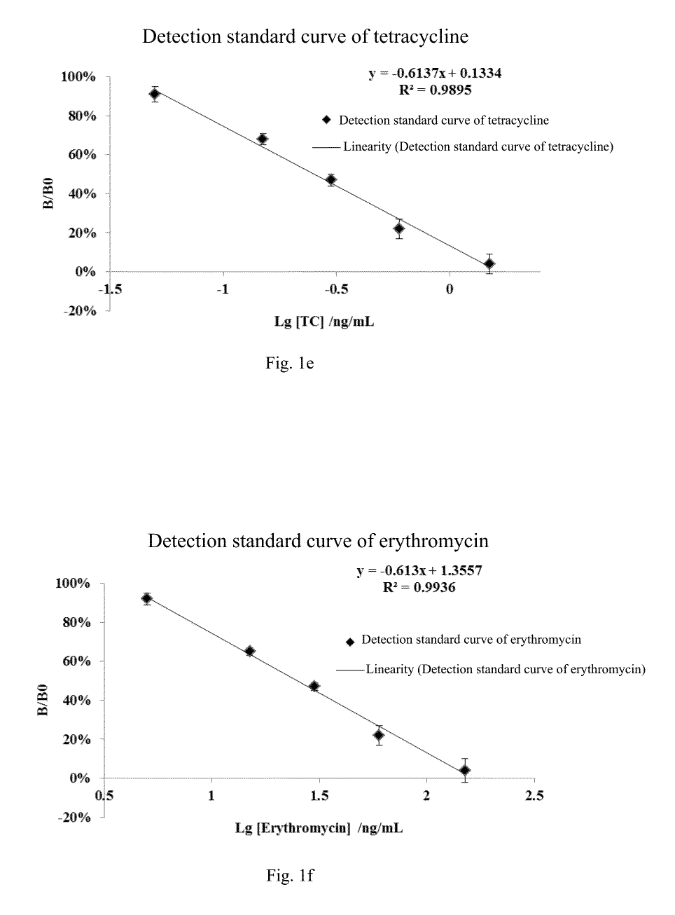

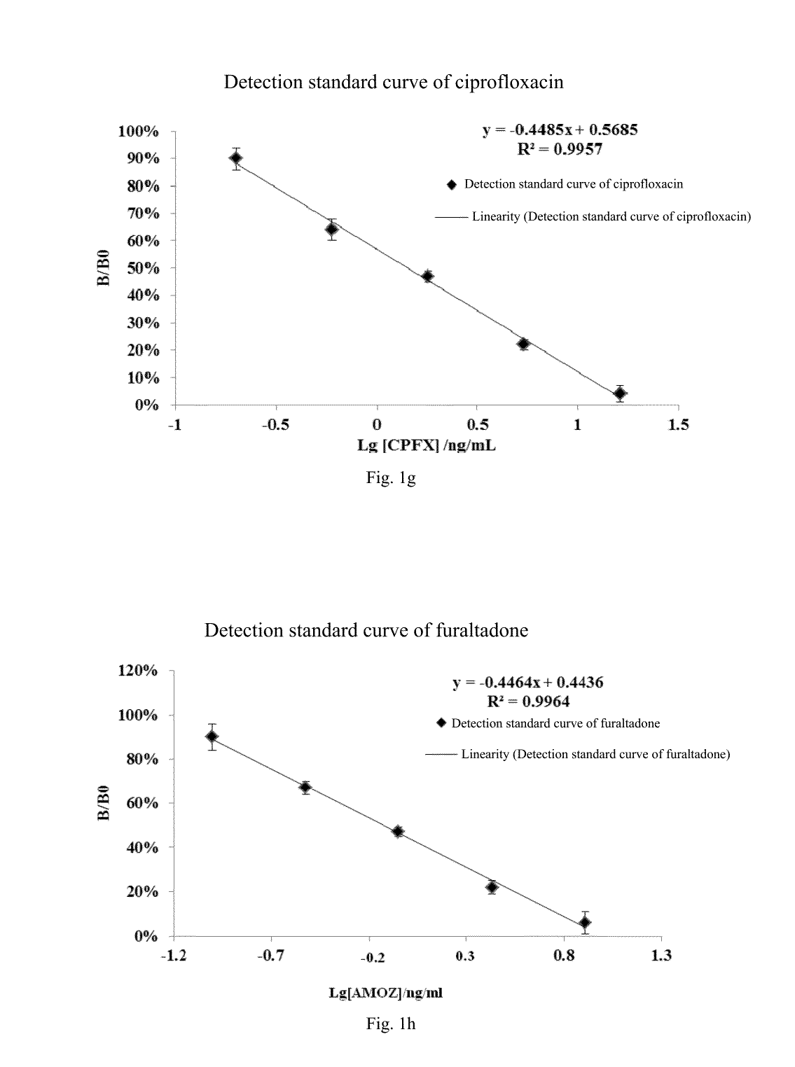

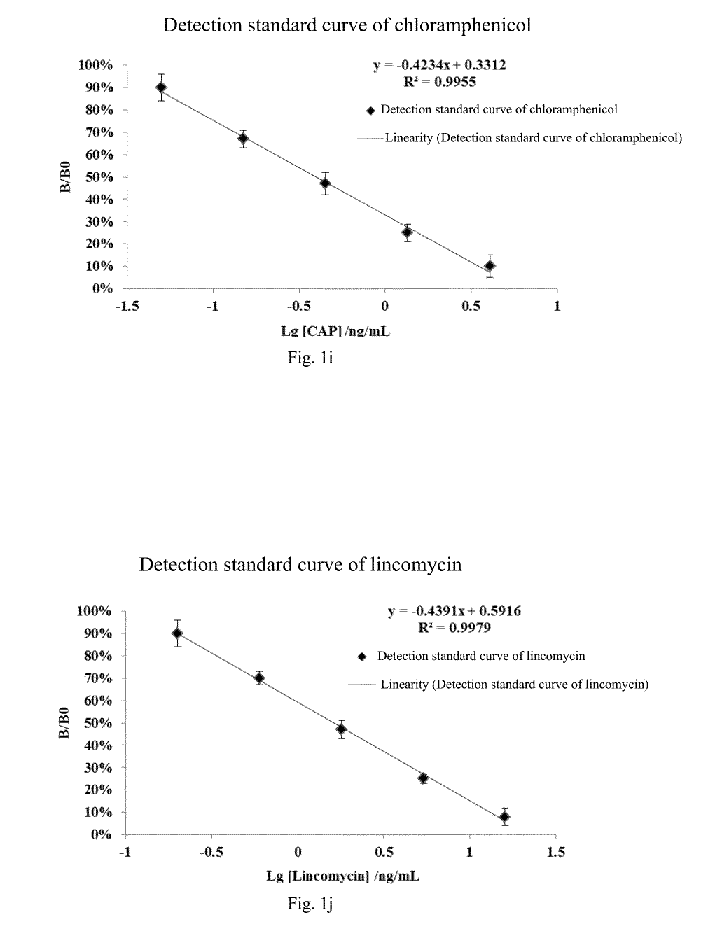

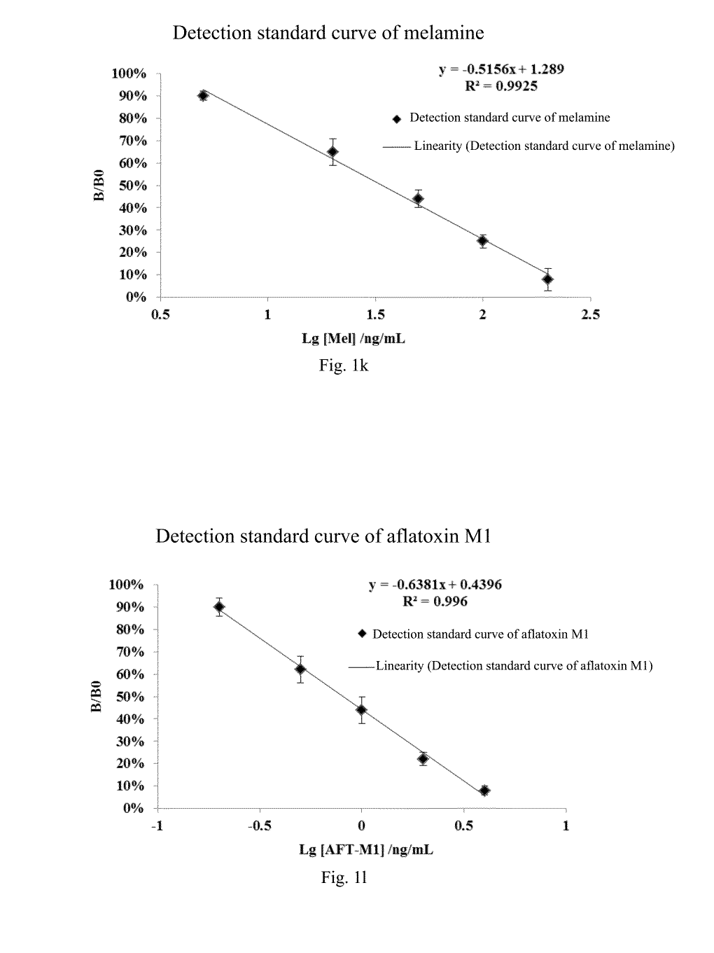

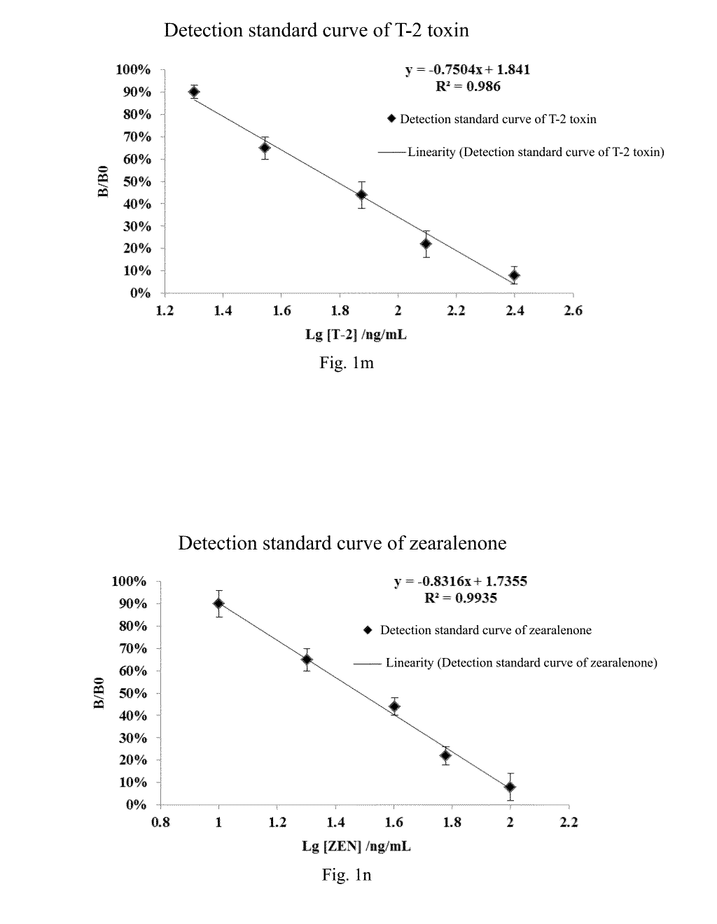

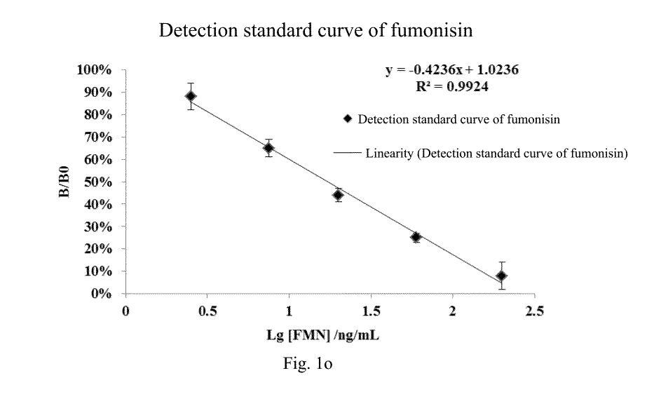

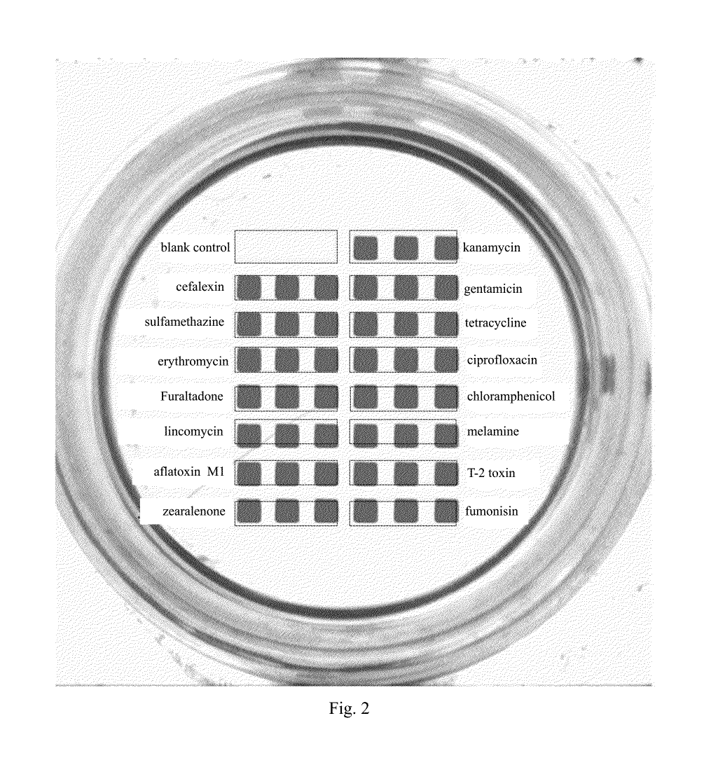

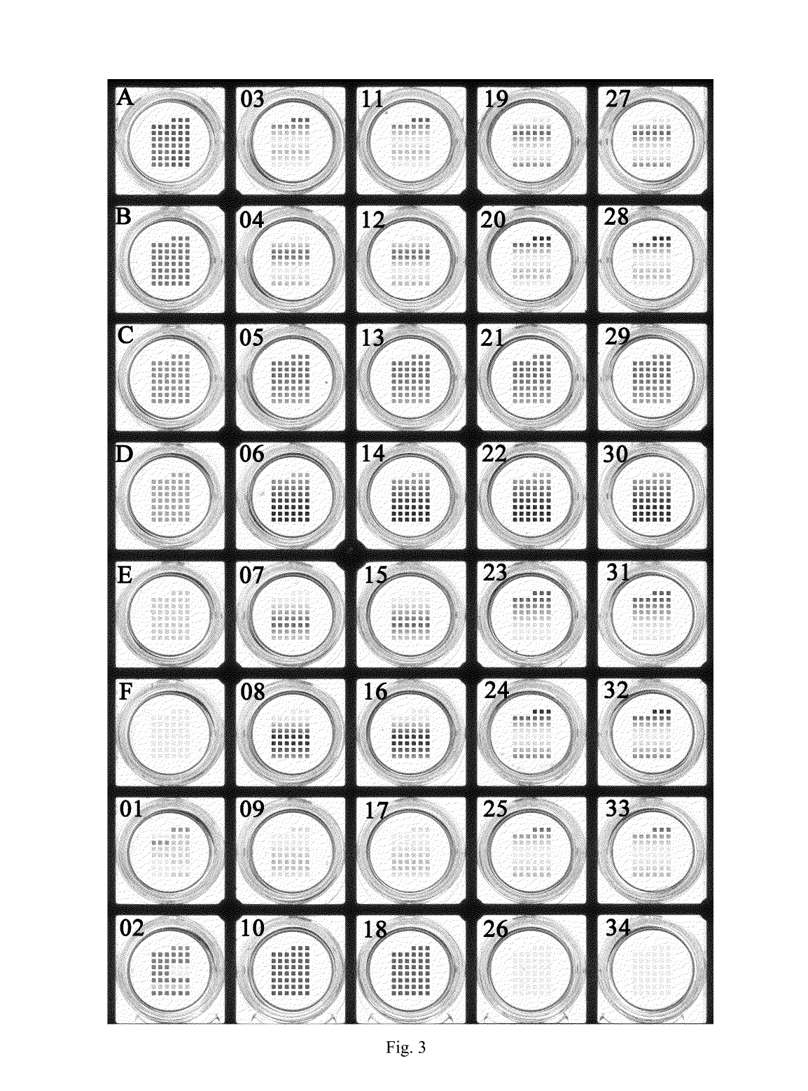

The detection results were as shown in FIGS. 1a-1o, FIG. 2 and FIG. 3.

The embodiment investigated the detection limits and the precision of the method. It could be seen from the figures that the quantitative limits of the method against detection targets were respectively as follows: the detection limit of kanamycin was 1 ng/mL, the detection limit of cefalexin was 2 ng/mL, the detection limit of gentamicin was 1 ng/mL, the detection limit of sulfamethazine was 0.8 ng/mL, the detection limit of tetracycline was 0.05 ng/mL, the detection limit of erythromycin was 5 ng/mL, the detection limit of ciprofloxacin was 0.2 ng/mL, the detection limit of furaltadone was 0.1 ng/mL, the detection limit of chloramphenicol was 0.05 ng/mL, the detection limit of lincomycin was 0.2 ng/mL, the detection limit of melamine was 5 ng/mL, the detection limit of aflatoxin M1 was 0.2 ng/mL, the detection limit of T-2 toxin was 20 ng/mL, the detection limit of zearalenone was 10 ng/mL and the detection limit of fumonisin was 2.5 ng/mL; and the method had relatively high precision: the CV value between wells was lower than 12% and the CV value in the wells was lower than 8%.

The embodiment investigated the accuracy, the precision and the like of the content detection method of kanamycin, cefalexin, gentamicin, sulfamethazine, tetracycline, erythromycin, ciprofloxacin, furaltadone, chloramphenicol, lincomycin, melamine, aflatoxin M1, T-2 toxin, zearalenone and fumonisin in the milk sample by using the method, and the results were as shown in the following table:

TABLE 1
Verification Results of Content Detection
Method of Various Substances in Milk Sample

| | | Correlation | | CV | Sample |
|---|---|---|---|---|---|
| | | coefficient | CV in | between | recovery |
| Detection target | Linear range | R² | wells | wells | rate |
| Kanamycin | 1 ng/mL-20 ng/mL | >0.99 | <12% | <8% | 90%-120% |
| Cefalexin | 2 ng/mL-50 ng/mL | >0.99 | <12% | <8% | 85%-105% |
| Gentamicin | 1 ng/mL-20 ng/mL | >0.99 | <12% | <8% | 87%-112% |
| Sulfamethazine | 0.8 ng/ml-20 ng/mL | >0.99 | <12% | <8% | 92%-109% |
| Tetracycline | 0.05 ng/ml-1.5 ng/mL | >0.99 | <12% | <8% | 85%-110% |
| Erythromycin | 5 ng/ml-150 ng/mL | >0.99 | <12% | <8% | 90%-110% |
| Ciprofloxacin | 0.2 ng/ml-16 ng/mL | >0.99 | <12% | <8% | 85%-115% |
| Furaltadone | 0.1 ng/ml-8 ng/mL | >0.99 | <12% | <8% | 80%-120% |
| Chloramphenicol | 0.05 ng/ml-4 ng/mL | >0.99 | <12% | <8% | 90%-120% |
| Lincomycin | 0.2 ng/ml-16 ng/mL | >0.99 | <12% | <8% | 85%-105% |
| Melamine | 5 ng/ml-200 ng/mL | >0.99 | <12% | <8% | 95%-105% |
| Aflatoxin M1 | 0.2 ng/ml-4 ng/mL | >0.99 | <12% | <8% | 90%-110% |
| T-2 | 20 ng/ml-250 ng/mL | >0.99 | <12% | <8% | 90%-112% |
| Zearalenone | 10 ng/ml-100 ng/mL | >0.99 | <12% | <8% | 85%-110% |
| Fumonisin | 2.5 ng/ml-200 ng/mL | >0.99 | <12% | <8% | 90%-112% |

The above results indicated that the biochip and the method provided by the invention were suitable for the detection of the actual samples, and the biochip and the method could also be utilized to detect other animal derived foods, including milk, milk powder, cheese, feed, urine, animal tissues (such as pork, beef, chicken, pork liver or chicken liver or the like), serum, honey, royal jelly, eggs and aquatic products (such as fish or shrimp or the like). Several representative animal derived foods were selected to further describe the invention below.

### Embodiment 2

The contents of gentamicin, tetracycline, terramycin, aureomycin, clarithromycin, sulfadiazine, norfloxacin, ciprofloxacin, ofloxacin, sudan red, trichothecenes and vomitoxin in the pork were simultaneously and quantitatively detected.

The detection steps were as follows:

(1) Preparation of a biochip: bovine serum albumin was taken as blank control, a biochip preparation instrument (US BioDot Company) was used to perform sample application on a 96-well plate to prepare a micro-array chip, bovine serum albumin, gentamicin, tetracycline, terramycin, aureomycin, clarithromycin, sulfadiazine, norfloxacin, ciprofloxacin, ofloxacin, sudan red, trichothecenes and vomitoxin antigens were subject to sample application operation on the 96-well plate, fixing was performed in a water bath at 20° C. for 4 h, a micro-array structure was formed on the 96-well plate, and array points in different positions corresponded to different antigens;

(2) Preparation of a solution of a sample to be detected: 5 g of crushed pork sample was taken, 8 mL of a buffer solution was added, mixing was performed for 5 min, then a mixture was put into the water bath at 50° C. for 30 min, centrifugation was performed for 10 min, 50 μL of supernatant liquid was taken, 450 μL of the buffer solution was added for mixing uniformly, and 50 μL was taken to obtain the solution of the sample to be detected;

(3) 10 μL of mixed standard solutions containing different concentration gradients of free gentamicin, tetracycline, terramycin, aureomycin, clarithromycin, sulfadiazine, norfloxacin, ciprofloxacin, ofloxacin, sudan red, trichothecenes and vomitoxin were respectively added into part of reaction wells of the 96-well plate, 10 μL of the solution of the sample to be detected was respectively added into each of the remaining reaction wells, 10 μL of monoclonal antibody mixed solutions corresponding to the gentamicin, tetracycline, terramycin, aureomycin, clarithromycin, sulfadiazine, norfloxacin, ciprofloxacin, ofloxacin, sudan red, trichothecenes and vomitoxin and 10 μL of horseradish peroxidase-labeled goat anti-mouse IgG were continuously added into the reaction wells added with the mixed standard solutions or the sample solution, reaction was performed in the water bath at 20° C. for 20 min and then washing was performed with a phosphate buffer solution;

(4) 10 μL of color developing agent and a substrate thereof were continuously added into each reaction well, the color developing agent comprised nanosilver and the substrate thereof, namely a chloroauric acid/hydrochinone system in the volume ratio of (1:1), the use was performed immediately after preparation, the color development reaction was performed for 5 min, and then the reaction was terminated;

(5) Image acquisition was performed by a CCD, and the contents of gentamicin, tetracycline, terramycin, aureomycin, clarithromycin, sulfadiazine, norfloxacin, ciprofloxacin, ofloxacin, sudan red, trichothecenes, vomitoxin and other substances in the sample were quantitatively detected by utilizing an external standard curve method according to array gray scale value signals in positions of coating antigens corresponding to the gentamicin, tetracycline, terramycin, aureomycin, clarithromycin, sulfadiazine, norfloxacin, ciprofloxacin, ofloxacin, sudan red, trichothecenes and vomitoxin.

### Embodiment 3

The contents of penicillin, streptomycin, terramycin, aureomycin, clarithromycin, sulfadiazine, sulfadimethoxine, norfloxacin, ofloxacin, furazolidone, thiamphenicol, malachite green and aflatoxin in shrimp meat were simultaneously and quantitatively detected.

(1) Preparation of a biochip: bovine serum albumin was taken as blank control, a biochip preparation instrument (US BioDot Company) was used to perform sample application on a 96-well plate to prepare a micro-array chip, bovine serum albumin, penicillin, streptomycin, terramycin, aureomycin, clarithromycin, sulfadiazine, sulfadimethoxine, norfloxacin, ofloxacin, furazolidone, thiamphenicol, malachite green and aflatoxin antigens were subject to sample application operation on the 96-well plate, fixing was performed in a water bath at 30° C. for 2 h, a micro-array structure was formed on the 96-well plate, and array points in different positions corresponded to different antigens;

(2) Preparation of a solution of a sample to be detected: 5 g of crushed shrimp meat sample was taken, 8 mL of a buffer solution was added, mixing was performed for 2 min, then a mixture was put into the water bath at 50° C. for 10 min, centrifugation was performed for 5 min, 50 μL of supernatant liquid was taken, 450 μL of the buffer solution was added for mixing uniformly, and 500 μL was taken to obtain the solution of the sample to be detected;

(3) 200 μL of mixed standard solutions containing different concentration gradients of free penicillin, streptomycin, terramycin, aureomycin, clarithromycin, sulfadiazine, sulfadimethoxine, norfloxacin, ofloxacin, furazolidone, thiamphenicol, malachite green and aflatoxin were respectively added into part of reaction wells of the 96-well plate, 200 μL of the solution of the sample to be detected was respectively added into each of the remaining reaction wells, 200 μL of monoclonal antibody mixed solutions corresponding to the penicillin, streptomycin, terramycin, aureomycin, clarithromycin, sulfadiazine, sulfadimethoxine, norfloxacin, ofloxacin, furazolidone, thiamphenicol, malachite green and aflatoxin and 200 μL of alkaline phosphatase-labeled goat anti-mouse IgG were continuously added into the reaction wells added with the mixed standard solutions or the sample solution, reaction was performed in the water bath at 30° C. for 10 min and then washing was performed with a Tris buffer solution;

(4) 100 μL of a color developing agent and a substrate thereof were continuously added into each reaction well, the color developing agent comprised horseradish peroxidase and the substrate thereof, namely, an o-phenylenediamine/hydrogen peroxide system in the volume ratio of (1:1), the use was performed immediately after preparation, the color development reaction was performed for 5 min, and then the reaction was terminated;

(5) Image acquisition was performed by a CCD, and the contents of penicillin, streptomycin, terramycin, aureomycin, clarithromycin, sulfadiazine, sulfadimethoxine, norfloxacin, ofloxacin, furazolidone, thiamphenicol, malachite green, aflatoxin and other substances in the sample were quantitatively detected by utilizing an external standard curve method according to array gray scale value signals in positions of coating antigens corresponding to the penicillin, streptomycin, terramycin, aureomycin, clarithromycin, sulfadiazine, sulfadimethoxine, norfloxacin, ofloxacin, furazolidone, thiamphenicol, malachite green and aflatoxin.

### Embodiment 4

The contents of penicillin, cefalexin, gentamicin, kanamycin, streptomycin, neomycin, tetracycline, terramycin, aureomycin, doxycycline, erythromycin, clarithromycin, roxithromycin, sulfadiazine, sulfamethazine, sulfamethoxazole, sulfadoxine, norfloxacin, ciprofloxacin, ofloxacin, gatifloxacin, sparfloxacin, moxifloxacin, furazolidone, furaltadone, nitrofurantoin, nitrofurazone, chloramphenicol, thiamphenicol, clindamycin and lincomycin in serum were simultaneously and quantitatively detected.

(1) Preparation of a biochip: bovine serum albumin was taken as blank control, a biochip preparation instrument (US BioDot Company) was used to perform sample application on a 96-well plate to prepare a micro-array chip, bovine serum albumin, penicillin, cefalexin, gentamicin, kanamycin, streptomycin, neomycin, tetracycline, terramycin, aureomycin, doxycycline, erythromycin, clarithromycin, roxithromycin, sulfadiazine, sulfamethazine, sulfamethoxazole, sulfadoxine, norfloxacin, ciprofloxacin, ofloxacin, gatifloxacin, sparfloxacin, moxifloxacin, furazolidone, furaltadone, nitrofurantoin, nitrofurazone, chloramphenicol, thiamphenicol, clindamycin and lincomycin antigens were subject to sample application operation on the 96-well plate, fixing was performed in a water bath at 37° C. for 0.5 h, a micro-array structure was formed on the 96-well plate, array points in different positions corresponded to different antigens; a 384-well plate, a transparent high-polymer chip base, a film or a glass sheet could also be adopted to replace the 96-well plate as a chip carrier;

(2) Preparation of a solution of a sample to be detected: 1 mL of serum sample was taken and placed in a 10 mL EP pipe, and diluted with a PBS buffer solution (0.1M, with pH of 7.4) for 100 times to obtain the solution of the sample to be detected;

(3) 50 μL of mixed standard solutions containing different concentration gradients of free penicillin, cefalexin, gentamicin, kanamycin, streptomycin, neomycin, tetracycline, terramycin, aureomycin, doxycycline, erythromycin, clarithromycin, roxithromycin, sulfadiazine, sulfamethazine, sulfamethoxazole, sulfadoxine, norfloxacin, ciprofloxacin, ofloxacin, gatifloxacin, sparfloxacin, moxifloxacin, furazolidone, furaltadone, nitrofurantoin, nitrofurazone, chloramphenicol, thiamphenicol, clindamycin and lincomycin were respectively added into part of reaction wells of the 96-well plate, 50 μL of the solution of the sample to be detected was respectively added into each of the remaining reaction wells, 50 μL of monoclonal antibody mixed solutions corresponding to the penicillin, cefalexin, gentamicin, kanamycin, streptomycin, neomycin, tetracycline, terramycin, aureomycin, doxycycline, erythromycin, clarithromycin, roxithromycin, sulfadiazine, sulfamethazine, sulfamethoxazole, sulfadoxine, norfloxacin, ciprofloxacin, ofloxacin, gatifloxacin, sparfloxacin, moxifloxacin, furazolidone, furaltadone, nitrofurantoin, nitrofurazone, chloramphenicol, thiamphenicol, clindamycin and lincomycin and 50 μL of nanogold particle-labeled goat anti-mouse IgG were continuously added into the reaction wells added with the mixed standard solutions or the sample solution, reaction was performed in the water bath at 37° C. for 20 min and then washing was performed with PBST;

(4) 50 μL of a color developing agent and a substrate thereof were continuously added into each reaction well, the color developing agent comprised horseradish peroxidase and the substrate thereof, namely, a tetramethylbenzidine/hydrogen peroxide system in the volume ratio of 1:1, the use was performed immediately after preparation, the color development reaction was performed for 5 min, and then the reaction was terminated;

(5) Image acquisition was performed by a CCD, and the contents of penicillin, cefalexin, gentamicin, kanamycin, streptomycin, neomycin, tetracycline, terramycin, aureomycin, doxycycline, erythromycin, clarithromycin, roxithromycin, sulfadiazine, sulfamethazine, sulfamethoxazole, sulfadoxine, norfloxacin, ciprofloxacin, ofloxacin, gatifloxacin, sparfloxacin, moxifloxacin, furazolidone, furaltadone, nitrofurantoin, nitrofurazone, chloramphenicol, thiamphenicol, clindamycin, lincomycin and other substances in the sample were quantitatively detected by utilizing an external standard curve method according to array gray scale value signals in positions of coating antigens corresponding to the penicillin, cefalexin, gentamicin, kanamycin, streptomycin, neomycin, tetracycline, terramycin, aureomycin, doxycycline, erythromycin, clarithromycin, roxithromycin, sulfadiazine, sulfamethazine, sulfamethoxazole, sulfadoxine, norfloxacin, ciprofloxacin, ofloxacin, gatifloxacin, sparfloxacin, moxifloxacin, furazolidone, furaltadone, nitrofurantoin, nitrofurazone, chloramphenicol, thiamphenicol, clindamycin and lincomycin.

### Embodiment 5

The contents of neomycin, doxycycline, roxithromycin, sulfamethoxazole, sulfadoxine, gatifloxacin, sparfloxacin, nitrofurantoin, clindamycin and botulinum toxin in honey were simultaneously and quantitatively detected.

(1) Preparation of a biochip: bovine serum albumin was taken as blank control, a biochip preparation instrument (US BioDot Company) was used to perform sample application on a 96-well plate to prepare a micro-array chip, bovine serum albumin, neomycin, doxycycline, roxithromycin, sulfamethoxazole, sulfadoxine, gatifloxacin, sparfloxacin, nitrofurantoin, clindamycin and botulinum toxin antigens were subject to sample application operation on the 96-well plate, fixing was performed in a water bath at 37° C. for 0.5 h, a micro-array structure was formed on the 96-well plate, array points in different positions corresponded to different antigens; a 384-well plate, a transparent high-polymer chip base, a film or a glass sheet could also be adopted to replace the 96-well plate as a chip carrier;

(2) Preparation of a solution of a sample to be detected: 50 μL of honey sample was taken and placed in a 10 mL EP pipe, and diluted with a PBS buffer solution (0.1M, with pH of 7.4) for 50 times to obtain the solution of the sample to be detected;

(3) 50 μL of mixed standard solutions containing different concentration gradients of free neomycin, doxycycline, roxithromycin, sulfamethoxazole, sulfadoxine, gatifloxacin, sparfloxacin, nitrofurantoin, clindamycin and botulinum toxin were respectively added into part of reaction wells of the 96-well plate, 50 μL of the solution of the sample to be detected was respectively added into each of the remaining reaction wells, 50 μL of monoclonal antibody mixed solutions corresponding to the neomycin, doxycycline, roxithromycin, sulfamethoxazole, sulfadoxine, gatifloxacin, sparfloxacin, nitrofurantoin, clindamycin and botulinum toxin and 25 μL of nanosilver-labeled goat anti-mouse IgG were continuously added into the reaction wells added with the mixed standard solutions or the sample solution, reaction was performed in the water bath at 37° C. for 20 min and then washing was performed with PBST;

(4) 50 μL of a color developing agent and a substrate thereof were continuously added into each reaction well, the color developing agent comprised alkaline phosphatase and the substrate thereof, namely, a BCIP/NBT system in the volume ratio of 1:1, the use was performed immediately after preparation, the color development reaction was performed for 5 min, and then the reaction was terminated;

(5) Image acquisition was performed by a CCD, and the contents of neomycin, doxycycline, roxithromycin, sulfamethoxazole, sulfadoxine, gatifloxacin, sparfloxacin, nitrofurantoin, clindamycin, botulinum toxin and other substances in the sample were quantitatively detected by utilizing an external standard curve method according to array gray scale value signals in positions of coating antigens corresponding to the neomycin, doxycycline, roxithromycin, sulfamethoxazole, sulfadoxine, gatifloxacin, sparfloxacin, nitrofurantoin, clindamycin and botulinum toxin.

### Embodiment 6

The contents of ochrotoxin, sterigmatocystin, citrinin, patulin, aflatoxins M1, M2, B1, B2, G1 and G2 in the feed were simultaneously and quantitatively detected.

The detection steps were as follows:

(1) Preparation of a biochip: bovine serum albumin was taken as blank control, a biochip preparation instrument (US BioDot Company) was used to perform sample application on a 96-well plate to prepare a micro-array chip, bovine serum albumin, ochrotoxin, sterigmatocystin, citrinin, patulin, aflatoxins M1, M2, B1, B2, G1 and G2 antigens were subject to sample application operation on the 96-well plate, fixing was performed in a water bath at 20° C. for 4 h, a micro-array structure was formed on the 96-well plate, and array points in different positions corresponded to different antigens;

(2) Preparation of a solution of a sample to be detected: 5 g of crushed feed sample was taken, 8 mL of a buffer solution was added, mixing was performed for 4 min, then a mixture was put into the water bath at 50° C. for 20 min, centrifugation was performed for 8 min, 50 μL of supernatant liquid was taken, 450 μL of the buffer solution was added for mixing uniformly, and 200 μL was taken to obtain the solution of the sample to be detected;

(3) 200 μL of mixed standard solutions containing different concentration gradients of free ochrotoxin, sterigmatocystin, citrinin, patulin, aflatoxins M1, M2, B1, B2, G1 and G2 were respectively added into part of reaction wells of the 96-well plate, 200 μL of the solution of the sample to be detected is respectively added into each of the remaining reaction wells, 200 μL of monoclonal antibody mixed solutions corresponding to the ochrotoxin, sterigmatocystin, citrinin, patulin, aflatoxins M1, M2, B1, B2, G1 and G2 and 200 μL of horseradish peroxidase-labeled goat anti-mouse IgG were continuously added into the reaction wells added with the mixed standard solutions or the sample solution, reaction was performed in the water bath at 20° C. for 20 min and then washing was performed with a phosphate buffer solution;

(4) 10 μL of a nano-color developing agent and a substrate thereof were continuously added into each reaction well, the color developing agent comprised nanogold and the substrate thereof, namely, 0.4M of HAuCl₄ and 0.1M of tannic acid in the volume ratio of 1:1, the use was performed immediately after preparation, the color development reaction was performed for 2 min, and then the reaction was terminated; (5) image acquisition was performed by a CCD, and the contents of ochrotoxin, sterigmatocystin, citrinin, patulin, aflatoxin M1, M2, B1, B2, G1 or G2 and other substances in the sample were quantitatively detected by utilizing an external standard curve method according to array gray scale value signals in positions of coating antigens corresponding to the ochrotoxin, sterigmatocystin, citrinin, patulin, aflatoxin M1, M2, B1, B2, G1 or G2.

### Embodiment 7

The contents of gentamicin, tetracycline, terramycin, aureomycin, clarithromycin and butenolide in the beef were simultaneously and quantitatively detected, and the operation was the same as that in embodiment 2.

1. A biochip for simultaneously detecting a variety of antibiotics, illegal additives and biotoxins in a visualized manner, characterized in that the biochip comprises a chip carrier fixed with a group of detection target antigens, the detection targets include the antibiotics, the illegal additives and the biotoxins, and the biochip is prepared by the following method: enabling the bovine serum albumin and the detection target antigens to perform sample application operation on the chip carrier through a biochip preparation system by taking bovine serum albumin as blank control, and then fixing in a water bath at 20-37° C. for 0.5-4 h for preparation.

2. The biochip for simultaneously detecting the variety of the antibiotics, the illegal additives and the biotoxins in the visualized manner according to claim 1, characterized in that the biochip is constituted by a group of matrices constituted by the detection target antigens, and the number of columns of each matrix is set to be a multiple of 3.

3. The biochip for simultaneously detecting the variety of the antibiotics, the illegal additives and the biotoxins in the visualized manner according to claim 1, characterized in that the chip carrier is a 96-well plate, a 384-well plate, a transparent high-polymer chip base, a film or a glass sheet.

4. The biochip for simultaneously detecting the variety of the antibiotics, the illegal additives and the biotoxins in the visualized manner according to claim 1, characterized in that the antibiotics are one or more of β-lactam antibiotics, aminoglycoside antibiotics, tetracycline antibiotics, macrolide antibiotics, sulfonamide antibiotics, quinolone antibiotics, nitrofuran antibiotics and other antibiotics; the illegal additives are one or more of melamine, malachite green and sudan red; and the biotoxins are one or more of aflatoxin, T-2 toxin, vomitoxin, zearalenone, fumonisin, ochrotoxin A, sterigmatocystin, butenolide, citrinin, patulin, trichothecenes and botulinum toxin.

5. The biochip for simultaneously detecting the variety of the antibiotics, the illegal additives and the biotoxins in the visualized manner according to claim 4, characterized in that the β-lactam antibiotics include penicillin antibiotics and cephalosporin antibiotics; the aminoglycoside antibiotics include gentamicin, kanamycin, streptomycin and neomycin; the tetracycline antibiotics include tetracycline, terramycin, aureomycin and doxycycline; the macrolide antibiotics include erythromycin, clarithromycin and roxithromycin; the sulfonamide antibiotics include sulfadiazine, sulfamethazine, sulfadimethoxine, sulfamethoxazole and sulfadoxine; the quinolone antibiotics include norfloxacin, ciprofloxacin, ofloxacin, gatifloxacin, sparfloxacin and moxifloxacin; the nitrofuran antibiotics include furazolidone, furaltadone, nitrofurantoin and nitrofurazone; and other antibiotics include chloramphenicol, thiamphenicol, clindamycin and lincomycin.

6. The biochip for simultaneously detecting the variety of the antibiotics, the illegal additives and the biotoxins in the visualized manner according to claim 4, characterized in that the aflatoxin is aflatoxin M1, M2, B1, B2, G1 or G2.

7. A method for simultaneously detecting a variety of antibiotics, illegal additives and biotoxins, characterized by comprising the following steps:
(1) respectively adding mixed standard solutions containing different concentration gradients of detection targets into part of reaction wells of a biochip, respectively adding a solution of a sample to be detected into the remaining reaction wells, further sequentially adding monoclonal antibodies and labeled goat anti-mouse IgG corresponding to the detection targets into all the reaction wells, reacting in a water bath at 20-37° C. for 10-20 min and washing with washing liquid;
(2) further adding a color developing agent into all the reaction wells, performing color development reaction for 2-5 min, and then terminating the reaction; and
(3) performing image acquisition by using CCD, making a standard curve according to gray scale values of antigens coated by the detection targets and concentration logarithms of the standard solutions, and respectively calculating the contents of the detection targets in the sample solution.

8. The method for simultaneously detecting the variety of the antibiotics, the illegal additives and the biotoxins in the visualized manner according to claim 7, characterized in that the sample to be detected is an animal derived food, including milk, milk powder, cheese, feed, urine, animal tissues, serum, honey, royal jelly, eggs and aquatic products.

9. The method for simultaneously detecting the variety of the antibiotics, the illegal additives and the biotoxins in the visualized manner according to claim 7, characterized in that the preparation method of the solution of the sample to be detected is as follows: when the sample to be detected is liquid, taking 50 μL-1 mL and putting into a 10 mL centrifuge tube, and diluting with a buffer solution for 10-100 times to obtain the solution of the sample to be detected; and when the sample to be detected is solid, taking 5 g of the homogeneous sample, adding 8 mL of the buffer solution, mixing for 2-5 min, putting into a water bath at 50° C. for 10-30 min, centrifuging for 5-10 min, taking 50 μL of supernatant liquid, adding 450 μL of the buffer solution, uniformly mixing, and taking 50 μL-500 μL to obtain the solution of the sample to be detected.

10. The method for simultaneously detecting the variety of the antibiotics, the illegal additives and the biotoxins in the visualized manner according to claim 7, characterized in that in step (1) the adding volume of the mixed standard solution of each detection target is 10-200 μL; the adding volume of the sample to be detected is the same as that of a standard solution of free antibiotics; the amount of the monoclonal antibody added in each reaction well is 10-200 μL; the amount of the labeled goat anti-mouse IgG added in each reaction well is 10-200 μL; the amounts of the monoclonal antibodies added in different reaction wells are the same, and the amounts of the labeled goat anti-mouse IgG added in different reaction wells are the same; and the labeled goat anti-mouse IgG is a nano-material or a biological enzyme-labeled goat anti-mouse IgG, and the washing liquid comprises a phosphate buffer solution and a Tris buffer solution.

11. The method for simultaneously detecting the variety of the antibiotics, the illegal additives and the biotoxins in the visualized manner according to claim 7, characterized in that, in step (2), the color developing agent comprises a biological catalyst and a substrate thereof or a chemical catalyst and the substrate thereof, and the adding volume of the color developing agent is 10-100 μL.
